(19) United States
(12) Patent Application Publication
(10) Pub. No.: US 2023/0023802 A1
(43) Pub. Date: Jan. 26, 2023

(54) STABILIZED DRUG FORMULATIONS AND METHODS OF LOADING DRUG DELIVERY IMPLANTS

(71) Applicant: THE METHODIST HOSPITAL SYSTEM, Houston, TX (US)

(72) Inventors: Antons Sizovs, Riga (LV); Fernanda Paola Pons Faudoa, Houston, TX (US); Alessandro Grattoni, Houston, TX (US)

(73) Assignee: THE METHODIST HOSPITAL SYSTEM, Houston, TX (US)

(21) Appl. No.: 17/756,859

(22) PCT Filed: Dec. 7, 2020
(86) PCT No.: PCT/US2020/063572

§ 371 Date: Jun. 3, 2022

Related U.S. Application Data
(60) Provisional application No. 62/943,993, filed on Dec. 5, 2019.
(60) Provisional application No. 62/961,294, filed on Jan. 15, 2020.

(51) Int. Cl. A61K 9/00; A61K 31/675; A61K 47/22; A61K 9/08

(52) U.S. Cl. CPC A61K 9/0024; A61K 31/675; A61K 47/22; A61K 9/08

(57) ABSTRACT

The present disclosure provides solid drug formulations, in particular to stabilized formulations of phosphonamidate-containing drugs, as well as methods for loading drug delivery devices with solid formulations.

### CROSS-REFERENCE TO RELATED APPLICATIONS

This application claims the benefit of priority to U.S. Provisional Application No. 62/943,993 filed Dec. 5, 2019, and U.S. Provisional Application No. 62/961,294, filed Jan. 15, 2020, the disclosure of each of which is incorporated herein by reference in its entirety.

### STATEMENT REGARDING FEDERALLY SPONSORED RESEARCH OR DEVELOPMENT

This invention was made with government support under Grant No. 5R01A1120749-03 awarded by the National Institutes of Health. The Government has certain rights in the invention.

### TECHNICAL FIELD

This disclosure relates to stabilized drug formulations and methods of loading drug delivery implants.

### BACKGROUND

Drug delivery implants have been used medically for a long time. The ability to load and refill drug delivery implants via a transcutaneous needle procedure is an essential requirement to extend the life of the implant and avoid surgical removal and replacement when the drug reservoir is fully depleted. Some refillable surgical implants are on the market, with more currently under development. Loading and refilling implants with a drug solution can be performed via needle injection. All of the currently available refillable implants can only be refilled with liquid compositions. However, many drugs are hydrophobic or insoluble at sufficiently high concentrations in the liquid solvents typically used in these applications. Additionally, many classes of drugs possess limited physicochemical stability in solution. One approach involves formulating the drug as an injectable suspension, but this may be limited by the requirement for a unique formulation for each drug, by the use of additional excipients, and/or by the implant capacity not being fully used.

Further, various therapeutic agents which possess phosphonamidate ester functional groups are of interest for the development of sustained and long-acting therapeutics for chronic pathologies and treatments. Due to their chemical structure, phosphonamidate esters have poor stability in aqueous solutions, limiting the development of long-acting formulations for depot- or reservoir-based delivery systems. One drug that contains a phosphonamidite ester functional group is tenofovir alafenamide fumarate (TAF), currently used in HIV pre-exposure prophylaxis (PrEP) and treatment as well as for the treatment of hepatitis B. Tenofovir is typically dosed orally with pills, but this is typically associated with poor adherence to the treatment regimen. Due to this, significant efforts have been put toward the development of long acting implantable or injectable formulations for the sustained delivery of TAF. However, TAF alone in a reservoir-based drug delivery implant or polymeric based long-acting formulation is not feasible due to rapid degradation of the drug's active components.

### SUMMARY

More specifically, the present disclosure provides methods for loading or refilling a drug delivery implant with a solid material directly without requiring surgical excision for the materials to be replaced.

In some aspects, disclosed is a method for loading or refilling a solid material into a drug delivery implant, wherein the drug delivery implant comprises a housing that defines an interior cavity, wherein the interior cavity is separated into a reservoir chamber and a filtrate chamber by a porous filter membrane, wherein the reservoir chamber comprises an inlet port, and wherein the filtrate chamber comprises an outlet port, the method comprising:

injecting a mixture into the reservoir chamber via the inlet port, wherein the mixture comprises a suspension of the solid material in a solvent, and wherein the porous filter membrane is permeable to the solvent but substantially impermeable to the solid material; and

removing the solvent from the filtrate chamber via the outlet port such that the solid material is retained within the drug delivery implant.

In some embodiments, the porous filter membrane has a porosity of 0.2 μm to 10 μm. The porous filter membrane may be made from a material such as a metal or metal alloy, glass, or a synthetic or natural polymer. In some embodiments, the porous filter membrane is physically attached within the interior cavity of the drug delivery implant, for example connected by welding, gluing, or fusing. In other embodiments, the porous filter membrane is removable.

In some embodiments, the mixture is injected into the inlet port with a first needle. In some embodiments, the solvent is removed via the outlet port with a second needle. In some embodiments, the inlet port and/or the outlet port comprise a self-sealing septum.

In some embodiments, the solid material has a solubility of less than 10 g/L in the solvent. In some embodiments, the solvent is an aqueous solution, for example phosphate buffered saline, an isotonic glucose solution, or Hank's balanced salt solution.

In another aspect, a method is provided for loading or refilling a solid material into a drug delivery implant, wherein the drug delivery implant comprises a housing that defines an internal cavity, wherein the internal cavity is separated into a reservoir chamber and a filtrate chamber by a porous filter membrane, wherein the filtrate chamber comprises an exterior port, and wherein the porous filter membrane comprises an interior port, the method comprising:

injecting a mixture through a first lumen into the reservoir chamber via the interior port, wherein the mixture comprises a suspension of the solid material in a solvent, and wherein the porous filter membrane is permeable to the solvent but substantially impermeable to the solid material; and

removing the solvent through a second lumen from the filtrate reservoir via the exterior port.

In some embodiments, the first lumen and the second lumen are both components of a single hypodermic needle.

In a further aspect, a method is provided for loading or refilling a solid material into a drug delivery implant, wherein the drug delivery implant comprises a housing that defines an internal cavity, and wherein the housing comprises an inlet port and an outlet port, the method comprising:

injecting a mixture via the inlet port into the internal cavity of the drug delivery implant, wherein the mixture comprises a suspension of the solid material in a solvent; and

removing the solvent from the internal cavity of the drug delivery implant via the outlet porting using a needle equipped with a porous filter membrane such that the solid material is retained within the drug delivery implant, wherein the porous filter membrane is permeable to the solvent but substantially impermeable to the solid material.

In some embodiments, the housing as found in any of the drug delivery implants disclosed herein may further comprise a semipermeable membrane that allows for controlled release of any active agent from the solid material as described herein. In some embodiments, the semi-permeable membrane is a nano-channeled membrane.

More specifically, a stabilized drug formulation is provided comprising an active agent containing at least one phosphonamidate ester group and a buffer, wherein the buffering agent has an aqueous solubility of less than 10 g/L. In some embodiments, the buffering agent has an aqueous solubility substantially similar to the aqueous solubility of the active agent, for example within 5% or within 10% of the aqueous solubility of the active agent. In some embodiments, the buffering agent has an aqueous solubility from 2 g/L to 10 g/L. In some embodiments, the active agent containing at least one phosphonamidate ester group is stable in the formulation for 30 days, 45 days, 50 days, 100 days, 150 days, 200 days, or more.

In some embodiments, the stabilized drug formulation comprises an active agent containing at least one phosphonamidate ester group and a buffering agent comprising urocanic acid. In some embodiments, the active agent and urocanic acid are present in substantially equal amounts by weight. In some embodiments, the active agent is selected from tenofovir alafenamide. In other embodiments, the buffering agent comprises phenylalanine, tyrosine, or isonicotinic acid.

In one aspect, the stabilized drug formulation described herein can be used in a long-acting drug delivery system. In some embodiments, the formulation can be used in a drug-releasing implant. In some embodiments, the formulation can be used in a long-acting drug formulation, such as a polymeric based formulation. In some embodiments, the formulation can be used in a drug-releasing film.

In another aspect, a method is provided for stabilizing an active agent containing at least one phosphonamidate ester group in a long-acting delivery system comprising combining the active agent with urocanic acid.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A is a schematic of a drug reservoir of an implant with two ports equipped with self-sealing septa. One of the ports (outlet) is separated from the rest of the drug reservoir by a filter permeable to liquids but poorly permeable or impermeable for suspended solid materials. FIG. 1B is a schematic showing an example using two needles for the inlet port and the outlet port. Once inside, the reservoir needs are separated by the filter. FIG. 1C is a schematic of the operating principle. A suspension of a drug (circles) is injected into the reservoir. Vehicle liquid of the drug suspension is removed through the outlet port. Solid drug cannot cross the filter and is accumulated inside the reservoir. FIG. 1D is a schematic showing the drug reservoir loaded with solid material when most of the vehicle fluid is removed. The resulting reservoir loading efficiency is very high.

FIG. 2 is a schematic showing examples of designs for filter incorporation. CS means the views are cross-sectional views.

FIG. 3 is an example of injector setup and the syringe for an implant design using a dual lumen needle.

FIG. 4 is a rendering of a sample implant as produced in Example 1.

FIG. 5 is a cross-sectional rendering of a sample implant as produced in Example 1.

FIG. 6A shows an empty top (lid) and bottom (reservoir) part of the sample implant of Example 1. The bottom part is equipped with a stainless steel cylindrical filter (0.5 μM porosity) tightly held between two rubber O-rings. FIG. 6B shows the assembled device with two needles connected to inlet and outlet ports. FIG. 6C shows the opened implant with solid barium sulfate loaded following the methods disclosed herein as described in Example 1.

FIG. 7A is a schematic of the pH stabilizing effect in a polymeric matrix containing undissolved drug particles; the low solubility buffer maintains long-term control of the pH locally despite the permeation of fluid from the body into the polymeric structure.

FIG. 7B is a schematic showing pH stabilization in a reservoir/membrane-based drug delivery implant.

FIG. 8 is a line graph showing the percentage of tenofovir alafenamide in the total mixture of tenofovir derivatives released from an implant loaded as described in Example 1 with three different tenofovir alafenamide formulations: tenofovir alafenamide fumarate (blue squares), free base tenofovir alafenamide (red triangles), and tenofovir alafenamide/urocanic acid formulations (green circles). Data are normalized to the values of day 1.

FIG. 9 is a bar graph showing the percentage of tenofovir alafenamide in the total mixture of tenofovir derivatives present in solution inside the reservoir loaded with the tenofovir alafenamide/urocanic acid as described in Example 2.

FIG. 10 is a schematic representation of the implant used for the procedures described in Example 3.

FIG. 11 is a schematic of the refilling setup used in Example 3. A sealed closed-loop system is provided that consists of: 1) a peristaltic pump; 2) a drug container (e.g., a flask) filled with a drug suspension and equipped with a Teflon-coated magnetic stirring bar; 3) a magnetic stirring plate; 4) tubing that connects the outlet port of the implant via a needle with the peristaltic pump and the drug container and which transfers the filtered solution; 5) tubing that connects the drug container with the inlet port of the implant via a needle and which transfers suspension; and 6) a pressure sensor.

FIG. 12 is a line graph that shows the dependence of the liquid transfer rate on the peristaltic pump speed as analyzed in Example 3.

FIG. 13A is a line graph showing the pressure changes during the in-vitro refilling procedure with UA suspension as described in Example 3.

FIG. 13B shows the UA deposited inside the implant during the refilling procedure described in Example 3.

FIG. 14 shows the in vivo experimental setup for refilling of drug implants in rats as described in Example 3.

FIG. 15 shows the positioned implant in the rat prior to refilling as described in Example 3.

FIG. 16 shows the in vivo solid refilling procedure performed on a rat as described in Example 3.

FIG. 17 shows the TA/UA loaded implant after the in vivo refilling procedure in a rat as described in Example 3.

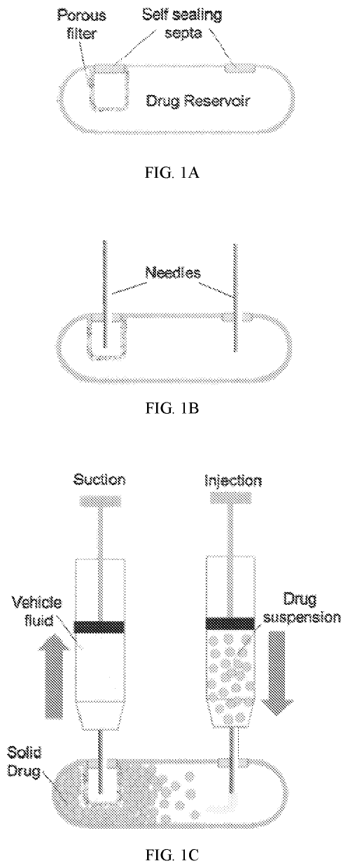

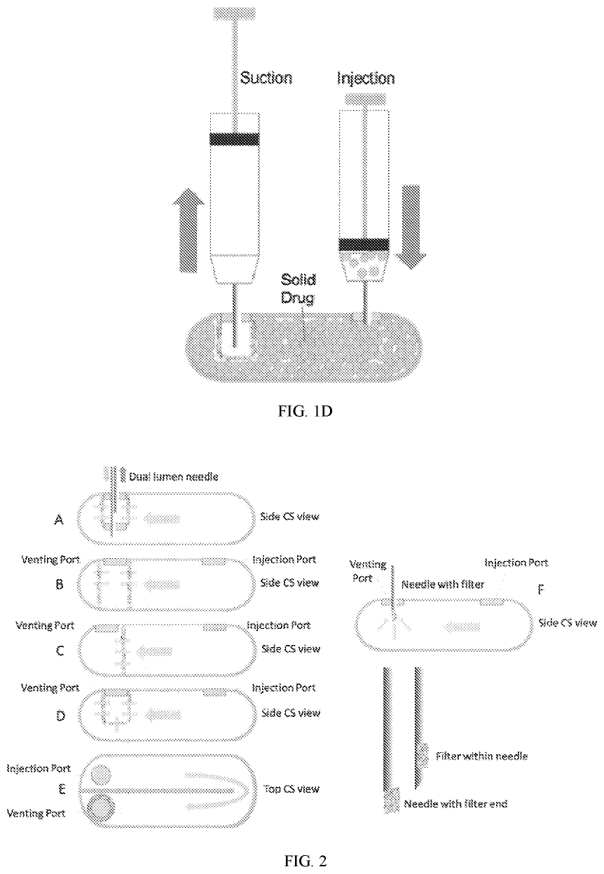

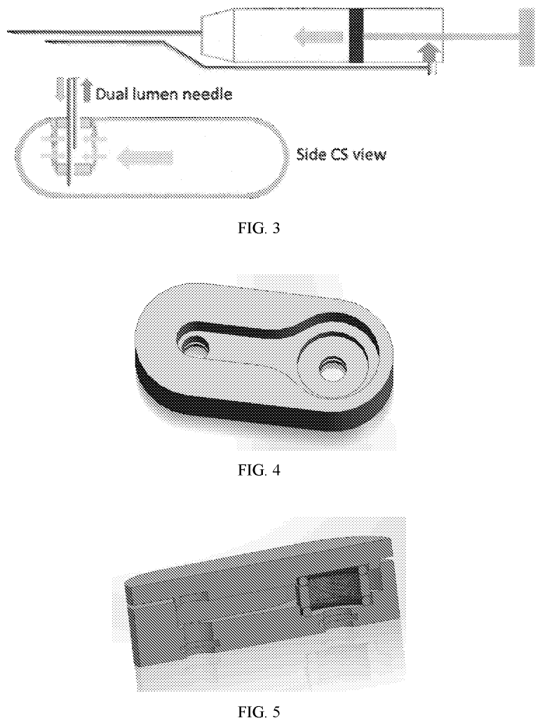

Like reference symbols in the various drawings indicate like elements.

### DETAILED DESCRIPTION

The devices, systems, materials, compounds, compositions, and methods described herein may be understood more readily by reference to the following detailed description of specific aspects of the disclosed subject matter and the Figures and Examples included therein.

Before the present devices, systems, materials, compounds, compositions, and methods are disclosed and described, it is to be understood that the aspects described below are not limited to specific methods or specific reagents, as such may, of course, vary. It is also to be understood that the terminology used herein is for the purposes of described particular aspects only and is not intended to be limiting.

Also, through this specification, various publications are referenced. The disclosures of these publications in their entireties are hereby incorporated by reference into this application in order to more fully described the state of the art to which the disclosed matter pertains. The references disclosed are also individually and specifically incorporated by reference herein for the material contained in the that is discussed in the sentence in which the reference is relied upon.

In this specification and in the claims that follow, reference will be made to a number of terms, which shall be defined to have the following meanings:

Throughout the specification and claims the word “comprise” and the other forms of the word, such as “comprising” and “comprises”, means including but not limited to, and is not intended to exclude, for example, other additive, components, integers, or steps.

As used in the description and the appended claims, the singular forms “a”, “an”, and “the” include plural references unless the context clearly dictate otherwise. Thus, for example, reference to “an inlet” includes mixtures of two or more such inlets, “a membrane” includes mixtures of two or more such membranes, and the like.

“Optional” or “optionally means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used.

By “reduce” or other forms of the word, such as “reducing” or “reduction”, is meant lowering of an event or characteristic. It is understood that this is typically in relation to some standard or expected value, in other words it is relative, but that is not always necessary for the standard or relative value to be referred to.

By “prevent” or other forms of the word, such as “preventing” or “prevention”, is meant to stop a particular event or characteristic, or to minimize the changes that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the other word is also expressly disclosed.

As used herein, “treatment” refers to obtaining beneficial or desired clinical results.

The term “patient” preferably refers to a human in need of treatment with one or more agents or treatments described herein for any purpose. However, the term “patient” can also refer to non-human animals, preferably mammals such as dogs, cats, horses, cows, pig, sheep and non-human primates, among others, that are in need of treatment with an agent or treatment described herein.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “composition” is intended to encompass a product comprising the specified ingredients in the specified amounts, as well as any product which results, directly or indirectly, from combination of the specified ingredients in the specified amounts.

As used herein, the term “device” is intended to encompass a product comprising the specific components, as well as any product which results, directly or indirectly, from combination of the specified components in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5 and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol. As described herein, “perfluoroalkyl” is an alkyl group as described herein where each hydrogen substituent on the group has been substituted with a fluorine atom. Representative but non-limiting examples of “perfluoroalkyl” groups include trifluoromethyl, pentafluoroethyl, or heptadecafluorooctyl.

The symbols Aⁿ is used herein as merely a generic substitutent in the definitions below.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Methods of Loading Drug Delivery Devices

The present disclosure is directed to methods and associated apparatuses for loading drugs that are solid materials into the reservoir of an implantable drug delivery device using needle injection. The disclosure provides a method and an associated port design that allows for the needle injection of a drug/liquid mixture in an implant and a separation of the liquid supernatant via filtration with resulting accumulation of a solid drug in the interior cavity of the implant. The filter can be incorporated inside the implant or alternatively can be incorporated into a needle. The disclosed approach allows loading or refilling of an implant with solid materials, including poorly soluble drugs.

Thus, in one aspect, a method is provided for loading a solid material into a drug delivery implant, wherein the drug delivery implant comprises a housing that defines an internal cavity, wherein the internal cavity is separated into a reservoir chamber and a filtrate chamber by a porous filter membrane, wherein the reservoir chamber comprises an inlet port, and wherein the filtrate chamber comprises an outlet port, the method comprising:

injecting a mixture into the reservoir chamber via the inlet port, wherein the mixture comprises a suspension of the solid material in a solvent, and wherein the porous filter membrane is permeable to the solvent but substantially impermeable to the solid material; and

removing the solvent from the filtrate chamber via the outlet port such that the solid material is retained within the drug delivery implant.

In another aspect, a method is provided for refilling a solid material into a drug delivery implant, wherein the drug delivery implant comprises a housing that defines an internal cavity, wherein the internal cavity is separated into a reservoir chamber and a filtrate chamber by a porous filter membrane, wherein the reservoir chamber comprises an inlet port, and wherein the filtrate chamber comprises an outlet port, the method comprising:

injecting a mixture into the reservoir chamber via the inlet port, wherein the mixture comprises a suspension of the solid material in a solvent, and wherein the porous filter membrane is permeable to the solvent but substantially impermeable to the solid material; and

removing the solvent from the filtrate chamber via the outlet port such that the solid material is retained within the drug delivery implant.

The drug-delivery implant can have any configuration or shape appropriate for maintaining biological activity and providing access delivery of a function, including for example, cylindrical, rectangular, disc-shaped, square-shaped, ovoid, stellate, or spherical. Moreover, the implant can be coiled or tubular. In cases where it is desired to retrieve the implant at a later time, configurations which tend to lead to migration of the implant from the site of implantation (such as spherical implant small enough to travel in the subject's blood vessels) should be avoided. In some embodiments, all or portions of the implant may be fabricated using a 3D printer. Thus, the shape can be highly complex and irregular, depending on the particular solid material and its location of use. Preferably, the implant can be configured to offer high structural integrity and easy retrieval from the subject. In some example, the implant is flexible so that it can be easily maneuvered.

The dimensions of the implant can be varied depending on the contents of the reservoir, the volume of the reservoir, the intended use, and the like. For example, the dimensions of the implant can permit serial implantation throughout a tissue volume via a minimally-invasive, trocar delivery mechanism. The dimensions can also be established to fit into a specific location in a subject. There are no strict requirements for the implant dimensions, and the implant can be ultimately tailored to match the size of commercially available deployment systems already adopted in clinics.

In some examples, the implant can have a diameter (or length) of less than 25 mm, for example 22 mm or less, 20 mm or less, 18 mm or less, 17 mm or less, 16 mm or less, 15 mm or less, 14 mm or less, 13 mm or less, or 12 mm or less. In other examples, the implant can have a diameter (or length) of 8 mm or greater, for example 9 mm or greater, 10 mm or greater, 11 mm or greater, 12 mm or greater, 13 mm or greater, 14 mm or greater, 14 mm or greater, 15 mm or greater, 16 mm or greater, 18 mm or greater, 20 mm or greater, 22 mm or greater, or 25 mm or greater. In some embodiments, the implant can have a diameter (or length) from 8 mm to 25 mm, from 10 mm to 25 mm, from 12 mm to 25 mm, or from 12 mm to 20 mm.

The height (or thickness) of the implant can be less than 8 mm, for example 7 mm or less, 6 mm or less, 5 mm or less, 4.5 mm or less, or 3 mm or less. In other embodiments, the height (or thickness) of the implant can be 2.5 mm or greater, for example 3 mm or greater, 4 mm or greater, 5 mm or greater, or 6 mm or greater. In some embodiments, the device can have a height (or thickness) of from 2.5 mm to 8 mm, from 3 mm to 8 mm, from 3 mm to 6 mm, or from 3.5 mm to 5 mm.

In some embodiments wherein the implant does not have a circular shape or diameter, the implant can have a longest linear dimension of less than 25 mm, for example 22 mm or less, 20 mm or less, 18 mm or less, 17 mm or less, 16 mm or less, 15 mm or less, 14 mm or less, 13 mm or less, or 12 mm or less. In other examples, the implant can have a longest linear dimension of 8 mm or greater, for example 9 mm or greater, 10 mm or greater, 11 mm or greater, 12 mm or greater, 13 mm or greater, 14 mm or greater, 14 mm or greater, 15 mm or greater, 16 mm or greater, 18 mm or greater, 20 mm or greater, 22 mm or greater, or 25 mm or greater. In some embodiments, the implant can have a longest linear dimension from 8 mm to 25 mm, from 10 mm to 25 mm, from 12 mm to 25 mm, or from 12 mm to 20 mm.

In some embodiments, the internal cavity of the drug delivery implant is separated into a reservoir chamber and a filtrate chamber by a porous filter membrane. The porous filter membrane can be fabricated from any material that may prove suitable for the purpose, i.e. a material fabricated in such a fashion as to be permeable to the solvent component of the mixture but that is substantially impermeable to the solid material. In some embodiments, the porous filter membrane comprises a metal or a metal alloy, including but not limited to titanium or steel. In some embodiments, the porous filter membrane may comprise a glass. In some embodiments, the porous filter membrane may comprise a polymer, including but not limited to a synthetic polymer such as polystyrene or a natural polymer such as cellulose.

In some embodiments, the porous filter membrane can have a porosity of 0.2 μm to 10 μm. In some embodiments, the filter membrane can have a porosity of 0.2 μm, 0.4 μm, 0.6 μm, 0.8 μm, 1.0 μm, 2.0 μm, 3.0 μm, 4.0 μm, 5.0 μm, 6.0 μm, 7.0 μm, 8.0 μm, 9.0 μm, or 10.0 μm. In some embodiments, the filter membrane can have a porosity from 0.2 to 10 μm, from 0.2 to 8 μm, from 0.2 to 6 μm, from 0.2 to 4 μm, from 0.2 to 4 μm, from 0.2 to 2 μm, from 0.2 to 1 μm, from 0.2 to 0.8 μm, from 0.2 to 0.4 μm, from 0.4 to 10 μm, from 0.4 to 8 μm, from 0.4 to 6 μm, from 0.4 to 4 μm, from 0.4 to 2 μm, from 0.4 to 1 μm, from 0.4 to 0.8 μm, from 0.8 to 10 μm, from 0.8 μm to 8 μm, from 0.8 μm to 6 μm, from 0.8 to 4 μm, from 0.8 to 2 μm, from 0.8 to 1 μm, from 1 to 10 μm, from 1 to 8 μm, from 1 to 6 μm, from 1 to 4 μm, from 1 to 2 μm, from 2 to 10 μm, from 2 to 8 μm, from 2 to 6 μm, from 2 to 4 μm, from 4 to 10 μm, from 4 to 8 μm, from 4 to 5 μm, from 6 to 10 μm, from 6 to 8 μm, or from 8 to 10 μm.

In some embodiments, the porous filter membrane can be physically attached within the interior cavity of the drug delivery implant. In some embodiments, the porous filter membrane may be incorporated by being directly connected to the drug delivery implant by an appropriate method such as welding, gluing, or fusing. In other embodiments, the porous filter membrane is removable.

The reservoir chamber of the implant as described herein can vary depending on the contents of the reservoir chamber, the volume of the reservoir chamber, the intended use, and the like. In some embodiments, the reservoir chamber can hold a volume from 10 μL to 200 μL, for example from 10 to 175 μL, from 10 to 150 μL, from 10 to 125 μL, from 10 to 100 μL, from 10 to 75 μL, from 10 to 50 μL, from 10 to 25 μL, from 25 to 175 μL, from 25 to 150 μL, from 25 t-125 μL, from 25 to 100 μL, from 25 to 75 μL, from 25 to 50 μL, from 50 to 175 μL, from 50 to 150 μL, from 50 to 125 μL, from 50 to 100 μL, from 50 to 75 μL, from 75 to 175 μL, from 75 to 150 μL, from 75 to 125 μL, from 75 to 100 μL, from 100 to 175 μL, from 100 to 150 μL, from 100 to 125 μL, from 125 to 175 μL, from 125 to 150 μL, from 150 to 175 μL, or from 175 to 200 μL.

The implant as used in the methods described herein can have at least one inlet port and at least one outlet port. In some embodiments, the inlet port is the point wherein the mixture containing the solid material is injected while the outlet port is the point of removal of the solvent component in the mixture, leading to deposition of the solid material within the implant. In some embodiments, the inlet port, the outlet port, or both are made from a material that is penetrable with a medical needle and resealable after penetration, e.g., a self-sealing septum. Such materials include plastic, rubber, or silicone. The needle may be inserted through the port for its intended purpose, wherein the port can be subsequently sealed. In some embodiments, the size of the inlet port or the outlet port can be from 0.5 mm to 3 mm, from 0.5 mm to 2 mm, or from 1 mm to 2 mm. in some embodiments, the inlet port or the outlet port can be accessed through the skin of the subject.

In some embodiments, the drug-delivery implant comprises a housing. The inclusion of a housing is to provide structure support to the components of the implant as used herein. The housing (or body) of the implant can be fabricated from a material that is biologically acceptable, e.g., does not illicit an immune response. Various polymers and polymer blends can be used to manufacture the device, including, biodegradable or non-biodegradable materials. The housing is preferably fabricated from a hydrophilic, viscoelastic, and/or biocompatible material. However, other materials can be used to fabricate the device and the surface of the device subsequently treated with a material that is hydrophilic, viscoelastic, and/or biocompatible. In specific examples, the device is surface treated with a biomaterial.

Examples of suitable materials for fabricating the housing include polylactic acids (PLA), polyalkylenes (including polypropylene and polyethylene), poly(alkylene glycols), polycarbonate (PC), cyclic olefin polymer (COP), poly(trimethylene carbonate), polycaprolactone (PCL), poly(lactic-co-glycolic acid) (PLGA), polyacrylates (including acrylic copolymers), polyacrylonitrile, polyvinylidenes, polyvinyl chloride copolymers, polyurethanes, polystyrenes, polyimides, polyamides, polethyleneimine, cellulose polymers (include cellulose acetates and cellulose nitrates), polysulfones (including polyethersulfones), polyesters, polyphosphazenes, poly(acrylonitrile-co-vinylchloride), poly(vinylsiloxane), as well as derivates, copolymers, and mixtures of the foregoing. Additional examples that may be used include polytetrafluoroethylene (PTFE), ePTFE (expanded polytetrafluoroethylene), hydroxypropyl methyl cellulose (HMPC), methacrylate polymers, poly(ethylene glycol), poly(ethyl ethacrylate), polyhydroxyvalerate, polyhydroxybutyrate, polydiaxanone, polyanhydrides, polycyanocrylates, poly(amino acids), poly(otheresters), copolymers of polyalkylene glycols, terephthalates, collagen, gelatin, chitosans, fibronectin, extracellular matrix proteins, vinculin, agar, agarose, alginates, derivatives thereof, or combinations thereof.

In some embodiments, the housing may further comprise a semi-permeable membrane. The semi-permeable membrane permits drugs, particles, and/or biomolecules to diffuse out from the reservoir of the implant once dissolved by the aqueous environment found within the body. In some embodiments, the semi-permeable membrane can be optimized for long-term release of drugs, particles, and/or biomolecules from the reservoir of the implant. As used herein, “controlled”, “sustained”, or “extended” release of the factors can be continuous or discontinuous, linear, or nonlinear. In some embodiments, the semi-permeable membrane can be made of silicon-containing materials or it can be a polymer like polyester, polycarbonate, poly(meth)methacrylate, or polylactic acid.

In some embodiments, the semipermeable membrane can be a nano-channeled membrane. Nano-channeled membranes are described in PCT/US2016/032658, which is incorporated herein by reference in its entirety. Briefly, the nano-channeled membrane can include hundreds of thousands of nano-channels with precisely controlled size and surface properties. At the nanoscale, molecular interactions within the channel wall dominate the transport of fluids to such an extent that the classical mechanical laws of diffusion (Fick's laws) break down. Thus, nanoscale phenomena are used herein to achieve the goal of constant release of bioactive agents over periods of times ranging from weeks to months and over a broad range of molecule sizes, at release rates relevant for medical applications. In some embodiments, the nano-channeled membrane can offer tightly-controlled release of drugs, particles, and/or biomolecules through its high spatial and electrostatic hindrance channels.

The nano-channels can be fabricated with varying height and channel density, enabling tuning to fit a given molecule and desired dose release rate. For example, the nano-channel membrane can have nanochannels from 2.5 nm to 1000 nm in diameter, for example from 2.5 nm to 750 nm, from 2.5 nm to 500 nm, from 2.5 nm to 100 nm, from 2.5 nm to 75 nm, from 2.5 nm to 50 nm, from 2.5 nm to 50 nm, from 2.5 nm to 25 nm, from 5 nm to 75 nm, from 5 nm to 50 nm, from 5 nm to 25 nm, from 10 nm to 75 nm, from 10 nm to 50 nm, from 10 nm to 25 nm, from 20 nm to 75 nm, from 20 nm to 50 nm, from 40 nm to 100 nm, from 40 nm to 75 nm, from 50 nm to 100 nm, rom 50 nm to 75 nm, from 75 nm to 100 nm, from 100 nm to 1000 nm, from 500 nm to 1000 nm, or from 750 nm to 1000 nm. The density of nano-channels in the semipermeable membrane can be at least 50,000, at least 100,000, or at least 150,000 nano-channels per mm².

The mixture as used in the methods described herein can comprise a solid material and a solvent. In preferred embodiments, the solid material comprises an active agent, such as a drug, and optionally one or more further excipients. The solid material is defined by having low solubility in the solvent component of the mixture. For example, the solid material may have a solubility in the solvent of less than 10 g/L, less than 9 g/L, less than 8 g/L, less than 7 g/L, less than 6 g/L, less than 5 g/L, less than 4 g/L, less than 3 g/L, less than 2 g/L, or less than 1 g/L.

The solvent component of the mixture as used in the methods herein can be any liquid component suitable for its intended purpose. Preferably, the solvent has limited toxicity such that it can be used in biological applications. In some embodiments, the solvent can be an aqueous solution. In some embodiments, the solvent is selected from phosphate buffered saline (PBS). In some embodiments, the solvent is an isotonic glucose solution. In some embodiments, the solvent is Hank's balanced salt solution.

The mixture may be injected into the reservoir chamber of the drug delivery implant via the inlet port using any method that may be suitable for the purpose. In some embodiments, the mixture is injected into the implant using a hypodermic needle or similar device such as a cannula. In other embodiments, the mixture may be injected via a pump.

The solvent may be removed from the filtrate chamber via the outlet port using any method that be suitable for the purpose. In some embodiments, the solvent may be removed using a hypodermic needle or similar device such as a cannula. In other embodiments, the solvent may be removed by a device that applies vacuum at the outlet port of the implant.

In yet other embodiments, the solvent may be removed from the reservoir by use of a filter syringe in addition to, or in place of, using an implant with a porous filter membrane contained therein. Filter syringes are known to those skilled in the art and are commercially available. In some embodiments, the porous filter membrane is contained within the need of the filter syringe. In other embodiments, the porous filter membrane is instead attached to a standard hypodermic needle as a separate component.

Thus, in another aspect, method is provided for loading or refilling a solid material into a drug delivery implant comprising an internal reservoir, wherein the drug delivery implant has an inlet port and an outlet port in communication with the internal reservoir, the method comprising:

preparing a mixture of the solid material within a solvent;

injecting the mixture via the inlet port into the internal reservoir of the drug delivery implant; and

removing the solvent from the internal reservoir of the drug delivery implant via the outlet port using a needle equipped with a porous filter membrane such that the solid material is retained within the drug delivery implant, wherein the porous filter membrane is permeable to the solvent but substantially impermeable to the solid material.

In yet other embodiments, the drug delivery implant may instead comprise a housing that defines an internal cavity, wherein the internal cavity is separated into a filtrate chamber and reservoir chamber by a porous filter membrane, wherein the filtrate chamber comprises an exterior port, and wherein the porous filter membrane comprises an interior port. In such applications a needle having a first lumen and a second lumen can be used, with the first lumen being of sufficiently more length than the second lumen such that the first lumen may be inserted past the interior port to be in contact with the reservoir chamber while the second lumen remains in contact with the filtrate chamber. The first lumen can be used for loading of the mixture described into the reservoir chamber while the second lumen is used for the concurrent removal of the solvent component via the exterior port.

Thus in another aspect, A method for loading a solid material into a drug delivery implant, wherein the drug delivery implant comprises a housing that defines an internal cavity, wherein the internal cavity is separated into a reservoir chamber and a filtrate chamber by a porous filter membrane, wherein the filtrate chamber comprises an exterior port, and wherein the porous filter membrane comprises an interior port, the method comprising:

injecting a mixture through a first lumen into the reservoir chamber via the interior port, wherein the mixture comprises a suspension of the solid material in a solvent, and wherein the porous filter membrane is permeable to the solvent but substantially impermeable to the solid material; and

removing the solvent through a second lumen from the filtrate reservoir via the exterior port.

### Stabilized Drug Formulations

The present disclosure provides stabilized drug formulations comprising an active agent having at least one phosphonamidate ester group and urocanic acid. The drug formulations described herein can stabilize drugs containing phosphonamidate ester groups that otherwise would readily degrade under physiological conditions. In some embodiments, the active agents described herein can be stabilized in the drug formulation for more than 30 days, for more than 40 days, for more than 50 days, for more than 100 days, for more than 150 days, for more than 200 days, or more.

In some embodiments, the active agent is present in an amount of 95 wt. %, 90 wt. %, 85 wt. %, 80 wt. %, 75 wt. %, 70 wt. %, 65 wt. %, 60 wt. %, 55 wt. %, 50 wt. %, 45 wt. %, 40 wt. %, 35 wt. %, 30 wt. %, 25 wt. %, 20 wt. %, 15 wt. %, 10 wt. %, or 5 wt. %, based upon the total weight of the drug formulation. In some embodiments, the active agent is present in an amount from 5 wt. % to 95 wt. %, from 10 wt. % to 90 wt. %, from 20 wt. % to 80 wt. %, from 30 wt. % to 70 wt. %, from 40 wt. % to 60 wt. %, from 45 wt. % to 55 wt. %, from 20 wt. % to 90 wt. %, from 30 wt. % to 90 wt. %, from 40 wt. % to 90 wt. %, from 45 wt. % to 90 wt. %, from 30 wt. % to 80 wt. %, from 40 wt. % to 8 wt. %, from 45 wt. % to 80 wt. %, from 40 wt. % to 70 wt. %, from 45 wt. % to 70 wt. %, or from 45 wt. % to 60 wt. %, based upon the total weight of the drug formulation.

In some embodiments, urocanic acid is present in an amount of 95 wt. %, 90 wt. %, 85 wt. %, 80 wt. %, 75 wt. %, 70 wt. %, 65 wt. %, 60 wt. %, 55 wt. %, 50 wt. %, 45 wt. %, 40 wt. %, 35 wt. %, 30 wt. %, 25 wt. %, 20 wt. %, 15 wt. %, 10 wt. %, or 5 wt. %, based upon the total weight of the drug formulation. In some embodiments, urocanic acid is present in an amount from 5 wt. % to 95 wt. %, from 10 wt. % to 90 wt. %, from 20 wt. % to 80 wt. %, from 30 wt. % to 70 wt. %, from 40 wt. % to 60 wt. %, from 45 wt. % to 55 wt. %, from 20 wt. % to 90 wt. %, from 30 wt. % to 90 wt. %, from 40 wt. % to 90 wt. %, from 45 wt. % to 90 wt. %, from 30 wt. % to 80 wt. %, from 40 wt. % to 8 wt. %, from 45 wt. % to 80 wt. %, from 40 wt. % to 70 wt. %, from 45 wt. % to 70 wt. %, or from 45 wt. % to 60 wt. %, based upon the total weight of the drug formulation.

In some embodiments, the active agent and urocanic are present in substantially the same weight percentage in the drug formulation based upon the total weight of the drug formulation.

A “phosphonamidate ester” group as described herein is a functional group represented generally by the formula A¹P(═O)(OA²)(NA³A⁴), wherein A¹ can be an alkyl, alkenyl, alkynyl, aryl, heteroaryl, cycloalkyl, cycloalkenyl, heterocycloalkyl, or hetocycloalkenyl group, A² can be alkyl, cycloalkyl, or aryl group, and A³ and A⁴ can be independently hydrogen, an alkyl, alkenyl, alkynyl, aryl, heteroaryl, cycloalkyl, cycloalkenyl, heterocycloalkyl, or heterocycloalkenyl group, each of which A¹, A², A³, or A⁴ may be optionally substituted with one or more substituents as allowed by valency.

Phosphonamidate esters are a class of molecules that find broad use as insecticides, acaricides, herbicides, phosphonamidate ester DNA analogues, and within biology and medicine. Phosphonamidate esters can act on their biological targets either directly or serve as prodrugs for the corresponding phosphonic acids. One of the major limitations of phosphonamidate esters is their hydrolytic instability. The hydrolysis that results in liberation of phosphonic acid derivatives proceeds fast at physiologically relevant pH values. This issue is especially relevant in the formulation of phosphonamidate esters for long-acting therapeutic approaches such as reservoir-based drug releasing implants, long-acting polymeric formulations, and implantable drug-releasing films.

Phosphonamidate esters undergo hydrolysis at all pH ranges. The hydrolysis rate reaches a minimum at the pH of 4.9-5.5. For example, the hydrolysis half-life (t_{1/2}) of tenofovir alafenamide at physiological pH of 7.4 is under 2 days, but at pH=5.3 the t_{1/2} is over 4 months. Maintaining the pH of fluids within this acidic range for extended periods of time in open systems such as inner space of implants, or near long-acting injectable formulations and implantable drug-releasing films is not trivial.

The use of typical buffers known to one skilled in the art (such as phosphate buffer saline, Hank's balanced buffer solution, etc.) is not suitable for long-acting drug delivery systems as described herein. These buffers have high solubility which will result in fast diffusion of the buffering molecules out of the implant or away from the injected long-acting formulation. In addition, high initial concentrations of a buffer will cause undesirable levels of osmotic pressure build up.

Thus, the present disclosure is also directed to stabilized drug formulations that contain a phosphonamidate ester active agent and a buffering agent having limited solubility (for example, less than 10 g/L) and that produce a solution pH upon dissolution that stabilizes the active agent. The low molarity of the resulting solutions does not produce excessive osmotic pressure. Once the neutralized buffer agent diffuses out of the inner compartment of an implant or away from an injected formulation or implanted film containing the stabilized drug formulation therein, new equivalents of the buffer agent will dissolve, maintaining the pH at the desired value. In one embodiment, the buffering agent comprises urocanic acid.

Thus in one aspect, a stabilized drug formulation is provided comprising an active agent having at least one phosphonamidate ester group and a buffering agent, wherein the buffering agent has an aqueous solubility of less than 10 g/L. Suitable buffering agents may comprise urocanic acid, phenylalanine, tyrosine, isonicotinic acid, or combinations thereof. In some embodiments, the buffering agent comprises phenylalanine. In some embodiments, the buffering agent comprises tyrosine. In some embodiments, the buffering agent comprises urocanic acid. In some embodiments, the buffering agent comprises isonicotinic acid.

In some embodiments, the buffering agent is present in an amount of 95 wt. %, 90 wt. %, 85 wt. %, 80 wt. %, 75 wt. %, 70 wt. %, 65 wt. %, 60 wt. %, 55 wt. %, 50 wt. %, 45 wt. %, 40 wt. %, 35 wt. %, 30 wt. %, 25 wt. %, 20 wt. %, 15 wt. %, 10 wt. %, or 5 wt. %, based upon the total weight of the drug formulation. In some embodiments, the buffering agent is present in an amount from 5 wt. % to 95 wt. %, from 10 wt. % to 90 wt. %, from 20 wt. % to 80 wt. %, from 30 wt. % to 70 wt. %, from 40 wt. % to 60 wt. %, from 45 wt. % to 55 wt. %, from 20 wt. % to 90 wt. %, from 30 wt. % to 90 wt. %, from 40 wt. % to 90 wt. %, from 45 wt. % to 90 wt. %, from 30 wt. % to 80 wt. %, from 40 wt. % to 8 wt. %, from 45 wt. % to 80 wt. %, from 40 wt. % to 70 wt. %, from 45 wt. % to 70 wt. %, or from 45 wt. % to 60 wt. %, based upon the total weight of the drug formulation.

In some embodiments, the buffering agent has an aqueous solubility from 2 g/L to 10 g/L, from 2 g/L to 8 g/L, from 2 g/L to 6 g/L, from 2 g/L to 4 g/L, from 4 g/L to 10 g/L, from 4 g/L to 8 g/L, from 4 g/L to 6 g/L, from 6 g/L to 10 g/L, from 6 g/L to 8 g/L, or from 8 g/L to 10 g/L.

In some embodiments, the buffering agent has an aqueous solubility substantially similar to the aqueous solubility of the active agent, for example within 5%, within 10%, or within 20% of the aqueous solubility of the active agent. In some embodiments, the buffering agent has an aqueous solubility within about 0.5%, within 1%, within 5%, within 10%, within 15%, or within 20% of the aqueous solubility of the active agent. In some embodiments, the buffering agent has an aqueous solubility within from 0.5% to 20%, from 1% to 20%, from 5% to 20%, from 10% to 20%, from 15% to 20%, from 0.5% to 15%, from 1% to 15%, from 5% to 15%, from 10% to 15%, from 0.5% to 10%, from 1% to 10%, from 5% to 10%, from 0.5% to 5%, or from 1% to 5% of the aqueous solubility of the active agent.

In some embodiments, the buffering agent produces a solution pH upon dissolution that stabilized the active agent. For example, in some embodiments, the buffering agent produces a solution pH upon dissolution of about 4.0, about 4.5, about 5.0, about 5.3, about 5.5, about 5.8, about 6.0, about 6.5, about 7.0, or about 7.5. In some embodiments, for example, the buffering agent produces a solution pH upon dissolution ranging from about 4.0 to 7.5, from about 4.5 to 7.5, from about 5.0 to 7.5, from about 5.3 to 7.5, from about 5.5 to 7.5, from about 5.8 to 7.5, from about 6.0 to 7.5, from about 6.5 to about 7.5, from about 7.0 to about 7.5, from about 4.0 to 7.0, from about 4.5 to 7.0, from about 5.0 to 7.0, from about 5.3 to 7.0, from about 5.5 to 7.0, from about 5.8 to 7.0, from about 6.0 to 7.0, from about 6.5 to about 7.0, from about 4.0 to 6.5, from about 4.5 to 6.5, from about 5.0 to 6.5, from about 5.3 to 6.5, from about 5.5 to 6.5, from about 5.8 to 6.5, from about 6.0 to 6.5, from about 4.0 to 6.0, from about 4.5 to 6.0, from about 5.0 to 6.0, from about 5.3 to 6.0, from about 5.5 to 6.0, from about 5.8 to 6.0, from about 4.0 to 5.8, from about 4.5 to 5.8, from about 5.0 to 5.8, from about 5.3 to 5.8, from about 5.5 to 5.8, from about 4.0 to 5.5, from about 4.5 to 5.5, from about 5.0 to 5.5, from about 5.3 to 5.5, from about 4.0 to 5.3, from about 4.5 to 5.3, from about 5.0 to 5.3, from about 4.0 to 5.0, from about 4.5 to about 5.0, and from about 4.0 to about 4.5.

In some embodiments, the active agent is tenofovir alafenamide having the structure:

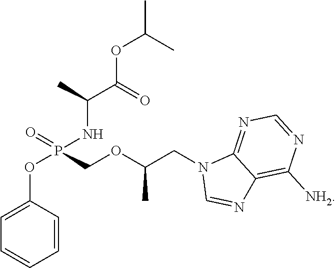 [SMILES: CC(C)OC(=O)[C@H](C)N[P@](=O)(CO[C@H](C)Cn1cnc2c(N)ncnc21)Oc1ccccc1]

Tenofovir alafenamide is used by itself or in combination with other antivirals (such as, for example, cobicistat, emtricitabine, elvitegravir, and/or darunavir) in the treatment of HIV and chronic hepatitis B and has potential for use in HIV pre-exposure prophylaxis due to the approval of the related tenofovir disoproxil fumarate for the same purpose. As treatment with these therapies often lasts for long periods of time, often months to years, stabilized drug formulations that can be used in systems that release the active agent over long periods, such as a drug implant, would be of a significant benefit.

In some embodiments, the active agent is ethyl ((((5-(6-amino-9H-purin-9-yl)-4-fluoro-2,5-dihydrofuran-2-yl)oxy)methyl)(phenoxy)phosphoryl)alaninate having the chemical structure:

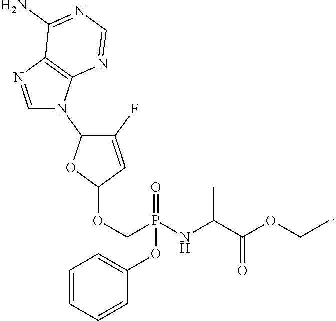 [SMILES: CCOC(=O)C(C)NP(=O)(COC1C=C(F)C(n2cnc3c(N)ncnc32)O1)Oc1ccccc1]

This compound is an example of an anti-viral inhibitory phosphonamidate ester compound and is described in U.S. Pat. Nos. 7,871,991 and 8,318,701, each of which is incorporated herein by reference in its entirety.

In some embodiments, the active agent is (phenethyl(phenoxy)phosphoryl)alanylproline having the chemical structure:

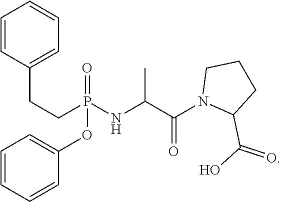 [SMILES: CC(NP(=O)(CCc1ccccc1)Oc1ccccc1)C(=O)N1CCCC1C(=O)O]

This compound is an angiotensin I converting enzyme (ACE) inhibitor and is described J. Med. Chem. 1985, 28(10):1422-1427, incorporated herein by reference in its entirety.

In some embodiments, the active agent is 4-fluorophenyl P-methyl-N-phenylphosphonamidate having the chemical structure:

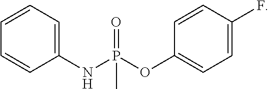 [SMILES: CP(=O)(Nc1ccccc1)Oc1ccc(F)cc1]

This compound is an inhibitor of DNA replication and HPGB (15-hydroxyprostaglandin dehydrogenase).

In some embodiments, the active agent is 4-isopropylphenyl-P-methyl-N-(4-((trifluoromethyl)thio)phenyl)phosphonamidate having the structure:

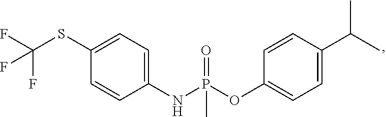 [SMILES: CC(C)c1ccc(OP(C)(=O)Nc2ccc(SC(F)(F)F)cc2)cc1]

This compound is an inhibitor of histone deacetylase 3 (HDAC3).

Further examples of active agents that may be used in the stabilized formulations disclosed herein are described in: Slusarczyk, M; Serpi, M.; and Pertusati, F. “Phosphoramidates and phosphonamidates (ProTides) with antiviral activity” Antivir. Chem. Chemother. 2018, 26:1-31; Pertusati, F.; Serpi, M.; and McGuigan, C. “Medicinal chemistry of nucleoside phosphonate prodrugs for antiviral therapy” Antivir. Chem. Chemother. 2012, 22:181-203; and Pradere, U. et al. “Synthesis of Nucleoside Phosphate and Phosphonate Prodrugs” Chem. Rev. 2014, 114, 9154-9218; each of which is incorporated herein by reference in its entirety.

### Long-Acting Drug Delivery Systems

The stabilized drug formulations described herein may be used in any drug delivery system where long-term dissolution and stability of the phosphonamidate active agent found therein is desired. The use of the stabilized drug formulation described herein in such systems ensures consistent delivery of these types of active agents over long periods without the need for frequent system replacement due to drug inactivation.

In some embodiments, the drug-delivery system is a drug-releasing implant. In some embodiments, the drug-releasing implant is as described in WO 2016/187100. In other embodiments, the drug-releasing implant is as described in WO 2019/079384.

In some embodiments, the drug delivery system is a polymeric-based formulation.

In some embodiments, the drug delivery system is a drug delivery film.

### Methods of Use

The stabilized drug formulations described herein and drug delivery devices thereto may be used in therapy against any disease or disorder for which the active agent is used.

In some embodiments, the disclosed stabilized drug formulations can be used in long-term drug delivery devices that are used to various therapeutic agents. Current methods of drug administration are associated with peaks and troughs of drug levels in the body. Such fluctuations affect drug efficacy and toxicities. Use of long-term drug delivery systems as described herein removes much of these wide swings and hence can allow the administration of specific drugs (at lower overall amounts) with fewer side effects without compromising efficacy. Other advantages are that the devices do not require any movable components and therefore represent a stable system which is less likely to suffer damage as opposed to osmotic pumps and electromechanical systems that sit outside the body. These advantages, combined with the aqueous stabilization of the described drug formulations for the phosphonamidate ester active agents combined therein, allow for decreased administration frequencies that may allow for greater compliance.

In one aspect, use of the stabilized formulations described herein in long-term drug delivery systems may prove useful for applications involving antiviral in the treatment or prophylaxis of HIV or related viral disease, particularly with tenofovir alafenamide (TAF). TAF is conventionally dosed daily by oral pills, which is often associated with poor patient adherence that can diminish outcomes. The use of a stabilized formulation with tenofovir alafenamide as described herein in a long-term drug delivery system, such as an implant, would diminish frequency of treatment administration to potentially a month or more, leading to possible higher compliance with the therapy.

Thus in some embodiments, a method for treating or preventing an infection with HIV in a subject is provided comprising administering the stabilized drug formulation including tenofovir alafenamide as described herein. In some embodiments, the administering step comprises implanting a drug-releasing implant comprising the stabilized drug formulation including tenofovir alafenamide as described herein. In some embodiments, the administering step comprising injecting a long-lasting polymeric-based formulation including tenofovir alafenamide as described herein. In other embodiments, the administering step comprises implanting a drug delivery film including tenofovir alafenamide as described herein.

In some embodiments, a method for treating or preventing an infection with Hepatitis B virus (HBV) in a subject is provided comprising administering the stabilized drug formulation including tenofovir alafenamide as described herein. In some embodiments, the administering step comprises implanting a drug-releasing implant comprising the stabilized drug formulation including tenofovir alafenamide as described herein. In some embodiments, the administering step comprising injecting a long-lasting polymeric-based formulation including tenofovir alafenamide as described herein. In other embodiments, the administering step comprises implanting a drug delivery film including tenofovir alafenamide as described herein.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The methods, associated devices, and associated compositions of the appended claims are not limited in scope by the specific methods and compositions described herein, which are intended as illustrations of a few aspects of the claims and any methods and associated compositions and devices that are functionally equivalent are within the scope of this disclosure. Various modifications of the methods and associated compositions and devices in addition to those shown and described herein are intended to fall within the scope of the appended claims. Further, while certain representative methods, and the compositions and devices associated therewith, are specifically described, other methods and combinations of various features of the methods are intended to fall within the scope of the appended claims, even if not specifically recited. Thus, a combination of steps, elements, components, and constituents can be explicitly mentioned herein; however, all other combinations of steps, elements, components and constituents are included, even though not explicitly stated.

### Example 1. Loading of Implant with Barium Sulfate

An implant was 3D printed with nylon and equipped with a stainless-steel cylindrical filter (0.5 μm porosity) tightly held between two rubber O-rings. Inlet and outlet ports were capped with silicone plus. A rendering of the implant is provided in FIG. 5.

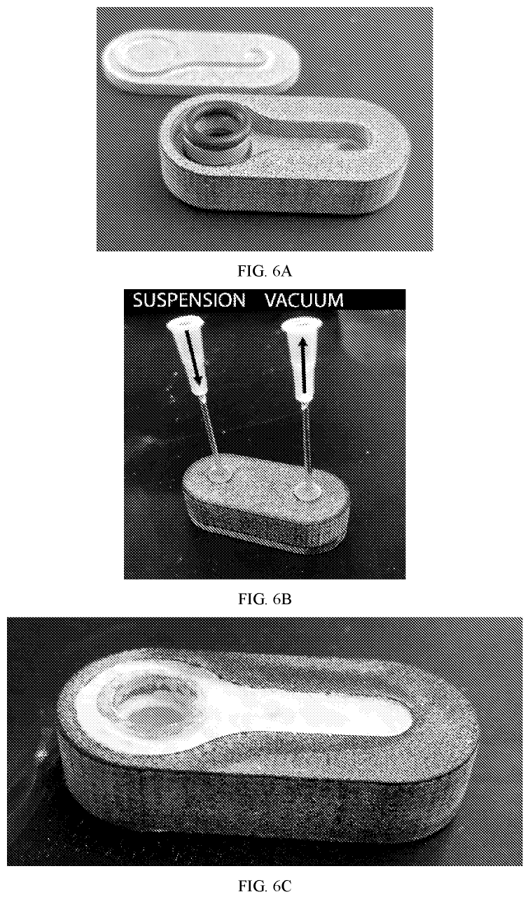

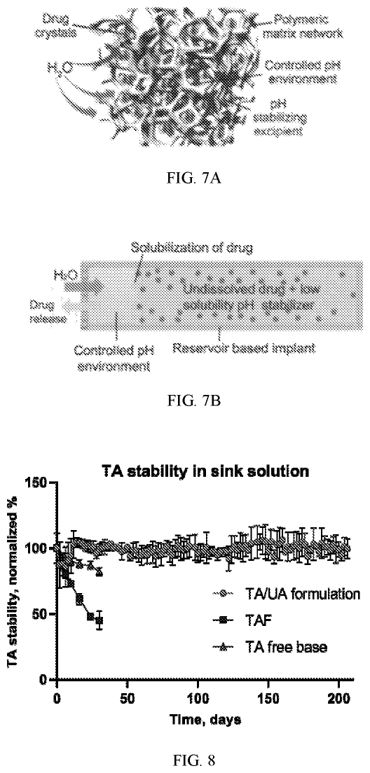

To demonstrate that solid material can be transported and deposited inside the implant, barium sulfate (BaSO₄) was chosen due to its poor solubility in water (approximately 2.5 μg/mL). The inlet port was connected to a suspension of barium sulfate in water that was continuously agitated; the outlet port was connected to a vacuum line. Filling of the device was continued until no more solid could be taken in. The lid was subsequently removed to reveal deposition solid material inside the reservoir. This process is demonstrated in FIG. 6.

### Example 2. Analysis of Stabilized Tenofovir Alafenamide Formulations in Drug Delivery Device

Formulations of tenofovir alafenamide with (i) monosodium dihydrogen phosphate/disodium hydrogen phosphate; (ii) phenylalanine; (iii) tyrosine); and (iv) urocanic acid were tested for their ability to maintain pH inside implantable devices equipped with nylon membranes with 200 nm porosity in vitro, alone with the ability of these formulations to release unhydrolyzed tenofovir alafenamide.

Formulation of tenofovir alafenamide (TA) with monosodium dihydrogen phosphate/disodium hydrogen phosphate was not able to able to maintain the pH below 6.3 and thus was not able to maintain the stability of TA.

In light of the challenges described above, molecules with lower water solubility but the same potential for controlling pH were considered. The amino acids L-phenylalanine and L-tyrosine were first tested. The solubility of L-tyrosine in a formulation with TA was not sufficient to provide pH buffering. The formulation with L-phenylalanine was able to maintain a pH of 5.8 inside the reservoir which was not sufficient to provide long-term stability for TA; in addition, L-phenylalanine displayed the tendency to precipitate on the membrane and clog the associated nanochannels.

Urocanic acid was then tested. The formulation was prepared by mixing equal masses of urocanic acid and tenofovir alafenamide free base. This formulation was loaded into an implant equipped with a 200 nm membrane. The implant was placed in 22 mL solution of PBS (the sink solution). The sink solution was replaced every 40 hours with fresh PBS. The sink solutions were analyzed using HPLC to determine the composition of the released formulation and determine the percentage of non-hydrolyzed tenofovir alafenamide. All results were normalized by the results of the first time point to compensate for the degradation that takes place in the sink solution. For comparison, the same experiments were conducted with implants loaded with tenofovir alafenamide fumarate (salt used in oral drug formulations) and with the free base tenofovir alafenamide.

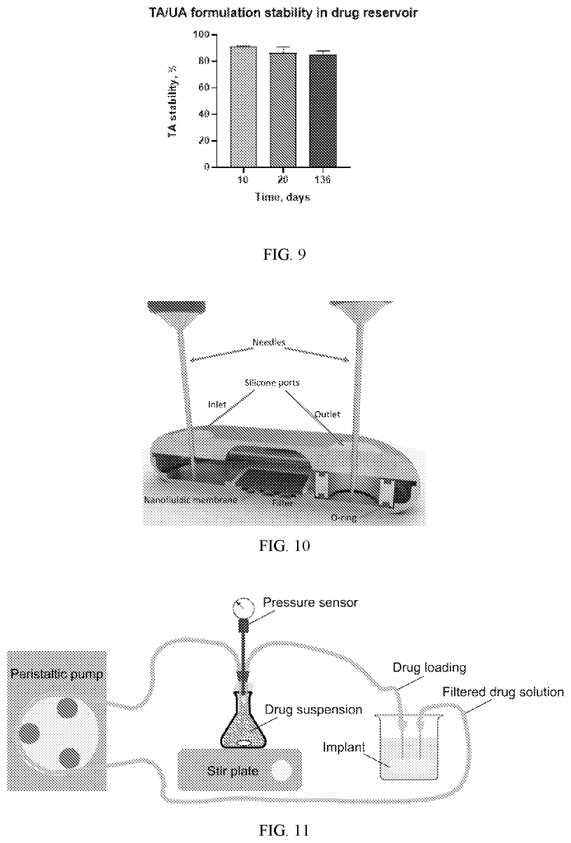

FIG. 8 demonstrates that the formulation with urocanic acid kept tenofovir alafenamide stable over a 200 day course. This is in contrast with free base tenofovir alafenamide loaded devices that after 1 month released a mixture of tenofovir derivatives with 82% tenofovir alafenamide, and in sharp contrast with tenofovir alafenamide fumarate that after 1 month released a mixture of tenofovir derivatives with one 45% tenofovir alafenamide. This increase in tenofovir stability, as measured by the percentage of tenofovir alafenamide found released in solution inside the reservoir among all tenofovir derivatives, was found to hold for at least over a 136 day course as shown in FIG. 9.

### Example 3. Transcutaneous Loading of Solid State Formulations

This present example describes the refilling of a urocanic acid+tenofovir alafenamide solid state formulation into an implantable reservoir.

### Implant

The implant (FIG. 10) features two self-sealing silicone ports that allow external access to the reservoir by means of needles. One port (outlet) is connected to the inside of a stainless steel tube that has a porous wall (the filter). The filter separates the outlet port from the rest of the reservoir to which the inlet port is connected. A solid suspension flows from the inlet port through the reservoir and gets filtered by the porous wall, depositing solid material in the reservoir and allowing the liquid phase to exit through the outlet port.

### Refilling Setup and General Considerations

A schematic of the refilling setup is provided in FIG. 11.

The operating principle for this procedure for loading non-dissolved solids into the implantable device parallels simple filtration. The setup must be a closed loop to minimize the amount of carrier solution required for the drug transfer. Carrier solution must be saturated with all materials present in the solids to avoid active dissolution and associated changes of the suspension physical properties during the refilling procedure. Length of the tubing leading to the inlet port must be minimal and the diameter of the tubing and needing must have no rapid changes in diameter (bottlenecks) to reduce potential changes of the suspension due to dissolution/precipitation processes.

### Rate of Transfer

The pressure inside the flask was continuously monitored using a pressure transducer connected to Arduino and a computer. In the set of experiments described, tubing with 1/16″ internal diameter was used, having a total volume of 2.28 mL connected to the implant with two microbore catheters (internal volume of 0.2 mL each).

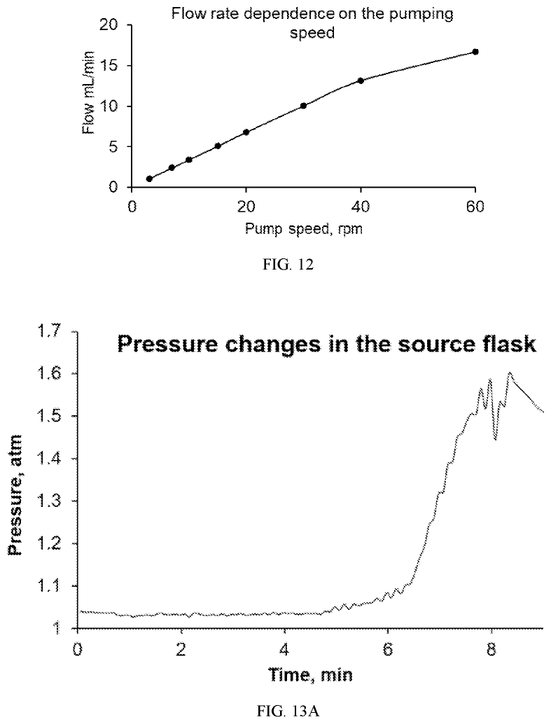

To establish the optimal flow rate, we tested the refilling setup at pump speeds ranging from 3-60 rpm using saline solution (see FIG. 12). Although the flow rate correlated linearly with the pump speed, bubble formation at the outlet port was observed for pump speeds above 7 rpm. Bubble formation is likely the result of transient pressure drops that lead to water boiling on the highly porous filter surface. Based on this result at a pump speed of 7 rpm, a flow rate of 2.5 mL/min which corresponds to that speed was used. At this flow rate, it takes approximately 14 seconds to displace the reservoir volume (0.57 mL).

### Urocanic Acid (UA) Suspension Preparation

To 500 mg of urocanic acid was added 11 mL of saline solution, and the mixture was homogenized with a tissue homogenizer at 26,000 rpm for 30 s. The suspension was first spun at 5000 rpm for 5 min, and a portion of the supernatant was collected and used to prime the tubing. The remaining suspension was re-homogenized, transferred to a flask equipped with a stirring bar and position on a stir plate continuously stirring throughout the refilling procedure.

### UA+TA (Tenofovir Alafenamide) Suspension Preparation

Urocanic acid (368.5 mg) and tenofovir alafenamide free base (327.0 mg) were mixed as solids and 11 mL of 0.9% saline solution was added. The obtained suspension was homogenized with a tissue homogenizer at 26,000 rpm for 30 seconds. The suspension was first spun at 5000 rpm for 5 min, and a portion of the supernatant was collected and used to prime the tubing. The remaining suspension was re-homogenized, transferred into a 5 mL flask equipped with a stirring bar and position on a stir plate and stirred continuously throughout the refilling procedure.

### UA Solid Refilling In Vitro

The implant was primed with saline solution using 23 G needles. The implant was the connected to the refilling setup using 21 G needles. The flask containing the UA suspension was capped with a septum and connected to the setup with 16 G needles. The implant was submerged in a beaker with saline solution. A stirring plate was set to 1000 rpm and the peristaltic pump at 2.4 mL/min (7 rpm). The experiment was repeated 3 times and after an average of 8 min, drug particles stopped flowing through the catheter which was accompanied by a rapid increase in pressure reading (see FIG. 13A). We define the refilling procedure to be complete when the pressure increased to approximately 1.6 atm. Z The implant was disconnected from the set up and opened to reveal that solid UA was occupying all available space (see FIG. 13B).

### UA+TA Solid Refilling In Vitro

The implant was primed with saline solution. The implant was connected to the refilling setup using 21 G needles, while the suspension-containing flask was connected with 16 G needles. The implant was submerged in a beaker with saline solution. The drug suspension was stirred at 1000 rpm and refilling was performed at a 2.4 mL/min flow rate. The refilling was stopped when the pressure of 1.6 atm was reached.

### UA+TA Solid Refilling In Vivo (Rat)

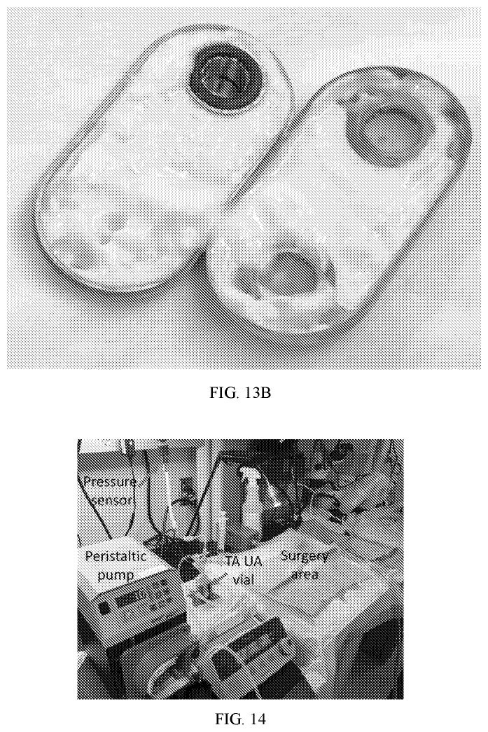

The refilling procedure established during the in vitro experiments was used to load TA/UA solid formulation into a device implanted in a rat. The implant was primed with saline solution under vacuum as previously described. Then, the implant was sterilized in 70% ethanol and placed in a tube with sterile saline solution. The in vivo experimental setup is shown in FIG. 14.

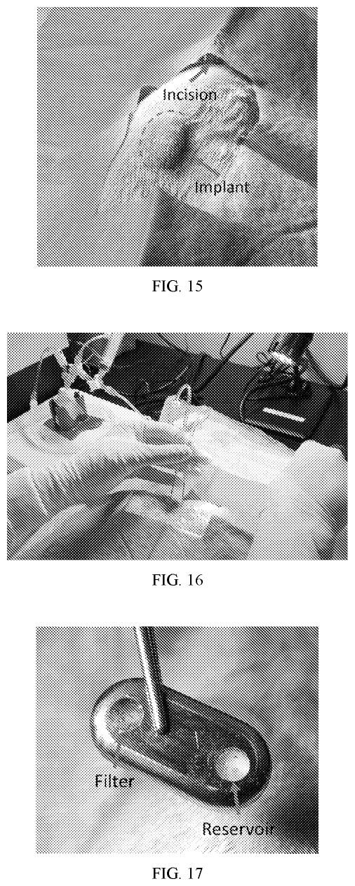

A Sprague Dawley rat was subcutaneously injected with buprenorphine 1 mg/kg. After 30 minutes, the rat was anesthetized with isoflurane. The rat's dorsum was trimmed and prepped for subcutaneous device implantation. With scissors, a subcutaneous incision subcervical on the dorsum was performed followed by a subcutaneous pocket on the lower right dorsum. The implant was inserted with the filter port caudal and with the membrane facing towards the fascia. Staples were used to close the wound which was cleaned afterwards with chloraprep swabs. See FIG. 15.

The implant was connected to the refilling loop using 18 G needles with the suspension flask was connected with 16 G needles. To insure correct implant positioning during the needle placement through the skin, it was stabilized with fingers underneath. With the other hand, each 18 G needle was inserted in its respective port. The stir plate was set to 1000 rpm and the peristaltic pump to 2.4 mL/min (7 rpm). The endpoint was reached after approximately 4 minutes when the pressure sensor marked 1.6 atm and drug particles stopped flowing through the tubing. Pumping was stopped, catheter connections were turned to disconnect the implant from the refilling setup, and needles were removed from the animal. See FIG. 16.

After the refilling procedure was complete, the implant was temporarily removed to make observations. Deposited solid material could be unmistakably seen through the silicone membrane of the inlet port, while the appearance of the outlet port membrane that is separated by filter from the rest of the implant remained unchanged. See FIG. 17.

### UA+TA Solid Refilling In Vivo (Non-Human Primate)

Indian rhesus macaques (macaca mulatta; n=4) were used to study the refilling procedure in non-human primates. All procedures were performed under anesthesia with ketamine (10 mg/kg, intramuscular) and phenytoin/pentobarbital (1 mL/10 lbs, intravenous). All animals had access to clean, fresh water at all times and a standard laboratory diet.

An approximately 1-cm dorsal skin incision was made on the right lateral side of the thoracic spine. Blunt dissection was used to make a subcutaneous pocket ventrally about 5 cm deep. The implant was placed into the pocket with the membrane facing the body. A simple interrupted tacking suture of 4.0 polydioxanone (PDS) was placed in the subcutaneous tissue to help closed the dead space and continued intradermally to close the skin. All animals received a single 50,000 U/kg perioperative penicillin G benzathine/penicillin G procaine (Combi Pen) injection and subcutaneous meloxicam (0.2 mg/kg). Upon completion of the surgery, the refilling procedure was performed.

To test the amount of drug deposited inside the implants, all drug-containing suspension and solution remaining was collected and quantified. Drug residual quantification was performed by dissolving all remaining solid drug in a known volume of HPLC grade water spiked with sodium azide. Obtained solutions were then analyzed by HPLC using a previously established protocol. The loading efficiency was 49.3±7/1% or 281±40 mg in an implant with a 570 μL reservoir as shown in Table 1.

TABLE 1
Loading Efficiency of Transdermal Refilling Procedure

| | Drug Formulation Loaded | % Loading |
|---|---|---|
| Macaque # | (mg) | Efficiency |
| M5 | 302 | 52.9% |
| M6 | 328 | 57.5% |
| M7 | 252 | 44.2% |
| M8 | 244 | 42.8% |
| Average + SD | 281 ± SD | 49.3 ± 7.1 |

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method for loading a solid material into a drug delivery implant, wherein the drug delivery implant comprises a housing that defines an internal cavity, wherein the internal cavity is separated into a reservoir chamber and a filtrate chamber by a porous filter membrane, wherein the reservoir chamber comprises an inlet port, and wherein the filtrate chamber comprises an outlet port, the method comprising:
injecting a mixture into the reservoir chamber via the inlet port, wherein the mixture comprises a suspension of the solid material in a solvent, and wherein the porous filter membrane is permeable to the solvent but substantially impermeable to the solid material; and
removing the solvent from the filtrate chamber via the outlet port such that the solid material is retained within the drug delivery implant.

2. A method for refilling a solid material into a drug delivery implant, wherein the drug delivery implant comprises a housing that defines an internal cavity, wherein the internal cavity is separated into a reservoir chamber and a filtrate chamber by a porous filter membrane, wherein the reservoir chamber comprises an inlet port, and wherein the filtrate chamber comprises an outlet port, the method comprising:
injecting a mixture into the reservoir chamber via the inlet port, wherein the mixture comprises a suspension of the solid material in a solvent, and wherein the porous filter membrane is permeable to the solvent but substantially impermeable to the solid material; and
removing the solvent from the filtrate chamber via the outlet port such that the solid material is retained within the drug delivery implant.

3. The method of any one of claim 1 or 2, wherein the porous filter membrane has a porosity of 0.2 μm to 10 μm.

4. The method of any one of claims 1-3, wherein the porous filter membrane comprises a metal or a metal alloy.

5. The method of claim 4, wherein the porous filter membrane comprises titanium.

6. The method of claim 4, wherein the porous filter membrane comprises steel.

7. The method of any one of claims 1-3, wherein the porous filter membrane comprises glass.

8. The method of any one of claims 1-3, wherein the porous filter membrane comprises a polymer.

9. The method of claim 8, wherein the porous filter membrane comprises polystyrene.

10. The method of claim 8, wherein the porous filter membrane comprises cellulose.

11. The method of any one of claims 1-10, wherein the mixture is injected into the inlet port with a first needle.

12. The method of any one of claims 1-11, wherein the solvent is removed via the outlet port with a second needle.

13. The method of any one of claims 1-12, wherein the inlet port comprises a self-sealing septum.

14. The method of any one of claims 1-13, wherein the outlet port comprises a self-sealing septum.

15. The method of any one of claims 1-13, wherein the solvent is an aqueous solution.

16. The method of claim 15, wherein the solvent is selected from phosphate buffered saline (PBS).

17. The method of claim 15, wherein the solvent is an isotonic glucose solution.

18. The method of claim 15, wherein the solvent is Hank's balanced salt solution.

19. A method for loading a solid material into a drug delivery implant, wherein the drug delivery implant comprises a housing that defines an internal cavity, wherein the internal cavity is separated into a reservoir chamber and a filtrate chamber by a porous filter membrane, wherein the filtrate chamber comprises an exterior port, and wherein the porous filter membrane comprises an interior port, the method comprising:
injecting a mixture through a first lumen into the reservoir chamber via the interior port, wherein the mixture comprises a suspension of the solid material in a solvent, and wherein the porous filter membrane is permeable to the solvent but substantially impermeable to the solid material; and
removing the solvent through a second lumen from the filtrate reservoir via the exterior port.

20. A method for loading a solid material into a drug delivery implant, wherein the drug delivery implant comprises a housing that defines an internal cavity, and wherein the housing comprises an inlet port and an out port, the method comprising:
injecting the mixture via the inlet port into the internal cavity of the drug delivery implant; and
removing the solvent from the internal cavity of the drug delivery implant via the outlet port using a needle equipped with a porous filter membrane such that the solid material is retained within the drug delivery implant, wherein the porous filter membrane is permeable to the solvent but substantially impermeable to the solid material.

21. The method of any one of claims 1-20, wherein the housing further comprises a semi-permeable membrane.

22. The method of claim 21, wherein the semi-permeable membrane comprises a nano-channeled membrane.

23. A stabilized drug formulation comprising an active agent having at least one phosphonamidate ester group and urocanic acid.

24. The drug formulation of claim 23, wherein the active agent is present in an amount of 95 wt. %, 90 wt. %, 85 wt. %, 80 wt. %, 75 wt. %, 70 wt. %, 65 wt. %, 60 wt. %, 55 wt. %, 50 wt. %, 45 wt. %, 40 wt. %, 35 wt. %, 30 wt. %, 25 wt. %, 20 wt. %, 15 wt. %, 10 wt. %, or 5 wt. %, based upon the total weight of the drug formulation.

25. The drug formulation of any one of claim 23 or 24, wherein urocanic acid is present in an amount of 95 wt. %, 90 wt. %, 85 wt. %, 80 wt. %, 75 wt. %, 70 wt. %, 65 wt. %, 60 wt. %, 55 wt. %, 50 wt. %, 45 wt. %, 40 wt. %, 35 wt. %, 30 wt. %, 25 wt. %, 20 wt. %, 15 wt. %, 10 wt. %, or 5 wt. %, based upon the total weight of the drug formulation.

26. The drug formulation of any one of claims 23-25, wherein the active agent and urocanic acid are present in substantially the same weight percentage in the drug formulation based upon the total weight of the drug formulation.

27. A stabilized drug formulation comprising an active agent having at least one phosphonamidate ester group and a buffer, wherein the buffer has an aqueous solubility of less than 10 g/L.

28. The stabilized drug formulation of claim 27, wherein the buffer includes urocanic acid.

29. The stabilized drug formulation of any one of claim 27 or 28, wherein the buffer includes phenylalanine.

30. The stabilized drug formulation of any one of claims 27-29, wherein the buffer includes tyrosine.

31. The stabilized drug formulation of any one of claims 27-30, wherein the buffer includes isonicotinic acid.

32. The stabilized drug formulation of any one of claims 27-31, wherein the buffering agent has an aqueous solubility within 5%, within 10%, or within 20% of the aqueous solubility of the active agent.

33. The drug formulation of any one of claims 23-32, wherein the active agent is tenofovir alafenamide having the chemical structure:

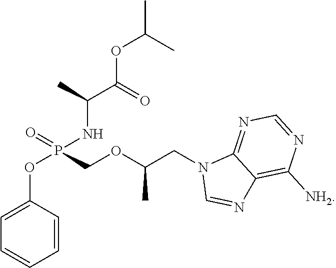 [SMILES: CC(C)OC(=O)[C@H](C)N[P@](=O)(CO[C@H](C)Cn1cnc2c(N)ncnc21)Oc1ccccc1]

34. The drug formulation of any one of claims 23-32, wherein the active agent is a compound having the chemical structure:

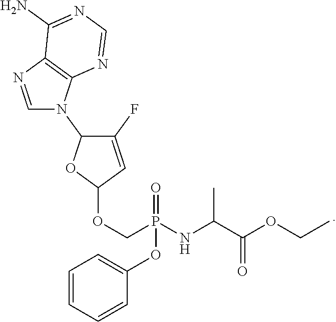 [SMILES: CCOC(=O)C(C)NP(=O)(COC1C=C(F)C(n2cnc3c(N)ncnc32)O1)Oc1ccccc1]

35. The drug formulation of any one of claims 23-32, the active agent is a compound having the chemical structure:

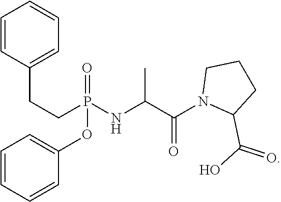 [SMILES: CC(NP(=O)(CCc1ccccc1)Oc1ccccc1)C(=O)N1CCCC1C(=O)O]

36. The drug formulation of any one of claims 23-32, the active agent is a compound having the chemical structure:

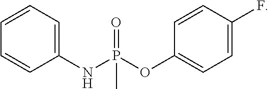 [SMILES: CP(=O)(Nc1ccccc1)Oc1ccc(F)cc1]

37. The drug formulation of any one of claims 23-32, the active agent is a compound having the chemical structure:

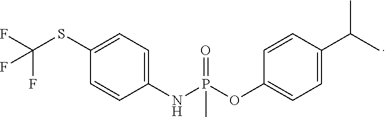 [SMILES: CC(C)c1ccc(OP(C)(=O)Nc2ccc(SC(F)(F)F)cc2)cc1]

38. A long-term drug delivery system comprising the drug formulation of any one of claims 23-37.

39. The drug delivery system of claim 38, wherein the drug delivery system is a drug implant.

40. The drug delivery system of claim 38, wherein the drug delivery system is a polymeric formulation.

41. The drug delivery system of claim 38, wherein the drug delivery system is a drug-releasing film.

42. A method for treating or preventing HIV in a subject comprising administering a stabilized drug formulation comprising tenofovir alafenamide and urocanic acid.

43. A method for treating or preventing HIV in a subject comprising implanting a drug implant, wherein the drug implant comprises a stabilized drug formulation comprising tenofovir alafenamide and urocanic acid.
